(19) United States
(12) Patent Application Publication
(10) Pub. No.: US 2022/0079188 A1
(43) Pub. Date: Mar. 17, 2022

(54) METHODS OF SAFELY DETERMINING AND DELIVERING AMOUNTS OF CANNABINOIDS IN ANIMALS

(71) Applicant: High Plains Nutrition, LLC, Inverness, FL (US)

(72) Inventor: Steven M. McGarrah, Inverness, FL (US)

(21) Appl. No.: 17/467,760

(22) Filed: Sep. 7, 2021

Related U.S. Application Data
(60) Provisional application No. 63/075,110, filed on Sep. 5, 2020.

(51) Int. Cl. A23K 20/111; A23K 40/25; A61B 5/00; A23K 50/40; A23K 10/30

(52) U.S. Cl. CPC A23K 20/111; A23K 40/25; A23K 10/30; A23K 50/40; A61B 5/4866

(57) ABSTRACT

A method for using an animal feed ration to safely stimulate the endocannabinoid system of an animal includes determining a daily metabolizable energy requirement (DMER) for a class of animals, such as a canine. Further, the method includes providing an animal feed ration meeting the daily metabolizable energy requirement (DMER) to at least one animal in said class of animals based on the body weight (BW) the said animals measurable in kilograms (kg). The method further includes feeding, or delivering, on a daily basis, the ration of food substrate to the mammalian subject in an amount of 2000 to 15000 kcal/kg of the animal subject BW, and the daily metabolizable energy requirement (DMER) is determined by the formula: DMER=RER_{MUL}*β (kgBW)^{-α}; where the maximum amount of daily cannabinoid intake provided is determined by the formula:

$$Maximum daily cannabinoid intake ( MDCI ) = \frac{D M E R}{R M E} \times PCI \times CC .$$

### FIELD OF THE INVENTION

The present invention relates to methods of feeding animal rations including cannabinoids in an amount related to Daily Metabolizable Energy Requirements (DMER) of the animal.

### BACKGROUND OF THE INVENTION

Cannabis Sativa L. has multiple beneficial effects on human and other mammalians. The bioactive molecules in Cannabis Sativa L are known to reduce inflammation, reduce tumors, reduce pain, and are known to have many other beneficial effects. On the contrary, there are limits to the amount of the major bioactive molecules yield beneficial effects, and in certain concentrations, the effects may be less than beneficial.

Animal feed rations are typically designed to have sufficient nutrition based on portion and the weight of the animal fed. The same reasoning applies to feed rations containing Cannabis Sativa L. Thus, it is ideal to have a quantity of bioactive cannabinoids that do not exceed a maximum threshold so that animals such as canine breeds, whether large or small, will not be adversely affected by the rations.

Much research and analysis has been performed to determine an ideal amount of Cannabis sativa L. that can be added to feed rations without having adverse effects on an animal subject regardless of size. This research indicates that cannabinoid content is to be managed, particularly the amounts of Cannabidiol (CBDA), which is the primary cannabinoid by concentration in the majority of strains of Cannabis sativa L., particularly in industrial hemp.

Cannabidiol has two major forms. The first is the naturally occurring and dominant acid form cannabinoid found in the Cannabis sativa L hemp strains in its natural state. This is CBDA, or cannabidiolic acid, which has the chemical formula of C₂₂H₃₀O₄, and a mass of 358.478 g/mol. The second is an oxidative product of CBDA, which is known generically as Cannabidiol or CBD, having the chemical formula of C₂₁H₃₀O₂, and a mass of 314.469 g/mol. In this document, the term Cannabidiol includes both CBDA and CBD unless otherwise specified.

Naturally, various cannabinoids accumulate in the flowering portions of the plant to protect the seeds from microbes, sunlight, and other adversity. It is natural for such cannabinoids to adhere to seed surfaces after the seed is separated from the flower/bud.

Cannabidiol can be found in the leaves, flowers and, to a lesser extent, the seeds of the Cannabis sativa L. hemp plant. The hemp seed oil has a Cannabidiol concentration due to naturally occurring Cannabidiol in the seeds, as well as contamination from other parts of the plant arising from cold pressing and other seed oil extraction methods. Rarely are the oil extraction processes refined enough to eliminate Cannabidiol that may be present as a contaminant, or coating on the seed biomass.

One benefit of the cold-pressing extraction process is that the acid form cannabinoids i.e. CBDA is preserved and not decarboxylated into the non-acid form molecule. It is beneficial, because the CBDA is understood to be more bioactive than the decarboxylated molecule (CBD).

Cannabinoids are oil-soluble molecules found in lipids extracted from hemp seeds and in the meal, seedcake, and protein powder products and byproducts. Excessive exposure to certain cannabinoids is harmful to animals. Many animal treats and supplements are commercially available that contain cannabinoids and have stated recommended doses for the pet owner or consumer to follow. However, none of these are known to incorporate any formulated controls that limit to the amount of cannabinoid exposure an animal may receive on a daily basis based on daily dietary feeding (incorporation into full feeding, life-sustaining daily diets), and some existing can harm animals if owners fail to precisely follow the stated instructions (for example, feeding excessive amounts of treats or increasing the recommended dosage).

Therefore, there is a need for preserving at least some of the cannabidiol (CBD) or cannabidiolic acid (CBDA) or both in an extruded pet food product, including hemp, particularly hemp seed products such as hemp meal, or hemp oil. Further, it is also desired a method of producing animal feed rations, such as extruded or pelletized animal food that is safe for consumption for animals of all sizes of any given species.

### SUMMARY OF THE INVENTION

Provided herein are the animal feed rations having cannabidiol (CBD) and cannabidiolic acid (CBDA) in a combined concentration determined in part by the daily metabolizable energy requirement (DMER), and said combined concentration being less than 0.504 mg/kg of a body weight (BW) of the mammalian subject, and said ratio of CBDA:CBD being at least 1:20 to achieve bioactivity such as a reduction in inflammation, tumor growth, pain, and other adverse biological issues.

In one aspect of the present invention provides a method for safe and effective incorporation of ingredients derived from the harvested seeds of the hemp (Cannabis Sativa L) plant by limiting the cannabinoid intake to less than 1.2 mg/kg of animal body weight (<1.2 mg/kgBW) per 24-hour period through animal feed ration (dietary formulation) incorporating mathematically calculated maximum exposures based on cannabinoid concentrations in the ingredients.

In one aspect of the present invention provides a method for using an animal feed ration to safely stimulate the endocannabinoid system of an animal, the method includes determining a daily metabolizable energy requirement (DMER) for a class of animals; providing an animal feed ration meeting the daily metabolizable energy requirement (DMER) to at least one animal in said class of animals, with at least one animal having a body weight (BW) measurable in kilograms (kg); the feed ration having cannabidiol (CBD) and cannabidiolic acid (CBDA) in a combined concentration determined in part by the daily metabolizable energy requirement (DMER), and said combined concentration being less than 0.504 mg/kg of the body weight of the animal, and said ratio of CBDA:CBD being at least 1:20; and delivering, on a daily basis, the feed ration to the animal in an amount of 2000 to 20000 kcal/kg of the body weight (BW) of the animal, wherein, the daily metabolizable energy requirement (DMER) is determined by a formula:

DMER=RER_{MUL}*β(kgBW)^{-α}

where,

DMER is the Daily Metabolizable Energy Required (in calories);

RER_{MUL} is a Resting Energy Required (RER);

kgBW is the animal body weight expressed in kilograms;

α is an adjustable factor having a value between 0.25 and 0.75;

β is a value between 65-6000; and

wherein, the maximum amount of combined cannabidiol (CBD) and cannabidiolic acid (CBDA) provided is determined by the formula:

$$Maximum daily cannabinoid intake ( MDCI ) = \frac{D M E R}{R M E} \times PCI \times CC$$

where,
DMER is the daily metabolizable energy requirement for the animal,
RME is the ration metabolizable energy per unit of mass (e.g., 3000 kcals/kg),
PCI is the percentage of cannabis ingredient in the ration on a dry weight (w/w) basis,
CC is the cannabinoid concentration in the cannabis ingredient on a dry weight basis.

In one aspect of the present invention, the combined CBD and CBDA concentration is greater than 0.1 mg/kg.

In one aspect of the present invention, the animal feed ration includes hemp oil that causes the CBD and CBDA concentration to be greater than 0.1 mg/kg.

In one aspect of the present invention, the animal feed ration includes hemp oil in a concentration of no more than 6% on a w/w basis in the animal feed ration.

In one aspect of the present invention, the ratio of CBDA:CBD in at least 1:20 and the animal feed ration is produced by extrusion.

In one aspect of the present invention, the ratio of CBDA:CBD in at least 1:20 and the animal feed ration is produced by pelletization.

In one aspect of the present invention, the animal feed ration is a canine feed ration having an absorbable energy of 2000-5000 kcal/kg

In one aspect of the present invention, the animal feed ration is equine feed ration having an absorbable energy of 10,000-15000 kcal/kg.

In one aspect of the present invention, the animal feed ration is feline feed ration having an absorbable energy of 2000-5000 kcal/kg.

In one aspect of the present invention, the animal fee ration has a ratio of CBDA:CBD of at least 1:20 to inhibit hepatoxicity in the at least one animal.

In one aspect of the present invention the class of animals is chosen from the group consisting of canines, equines, felines, or other mammalians and further subdivided by age.

In one aspect of the present invention the class of animals is further defined by breed, class, age, size, body condition or other factors.

In another aspect of the present invention provides a method for manufacturing extruded animal food having safe levels of cannabinoids, the method is providing a food substrate having a cannabinoid content of a combined cannabidiol (CBD) and cannabidiolic acid (CBDA) concentration of less than 5 mg/kg of the food substrate; extruding the substrate at a maximum temperature of no more than 145° C. and a pressure of no more than 50 kg/cm²; and cooling the extruded substrate to ambient temperature, wherein the extruded substrate is animal food having a moisture content of less than 35%, a bulk density of between 150-600 g/l, and at least 3% of the total Cannabinoid content is the cannabidiolic acid (CBDA).

In another aspect of the present invention, no isolated cannabidiol or cannabidiolic acid is added to the food substrate before or after the step of cooling.

In another aspect of the present invention, no distilled cannabidiol or cannabidiolic acid is added to the food substrate before or after the step of cooling.

In another aspect of the present invention no concentrated cannabidiol or cannabidiolic acid is added to the food substrate before or after the step of cooling.

In another aspect of the present invention, the animal food is achieved through formulation computations to limit the daily intake and exposure to any individual cannabinoid analyte to a maximum of 1.5 milligrams per kilogram of body weight per day (mg/kg BW/day) in animal food.

In another aspect of the present invention, the formulation limits the daily intake and exposure to any total potential decarboxylated cannabinoid analyte group (e.g., CBC, CBD, CBDV, CBG, CBL, CBN, THC, THCV) to a maximum of 1.5 milligrams per kilogram of body weight per day (mg/kg BW/day) in animal feeds.

In another aspect of the present invention, the cannabinoid includes two or more of CBCA, CBC, CBDA, CBD, CBDVA, CBDV, CBGA, CBG, CBL, CBNA, CBN, THCA, THC, THCVA, and THCV, and at least 3% of the total cannabinoid mass (in mg/kg or μg/g) of the sum of CBCA, CBC, CBDA, CBD, CBDVA, CBDV, CBGA, CBG, CBL, CBNA, CBN, THCA, THC, THCVA, and THCV consists of the cannabinoid CBDA in the manufactured animal feed or human food product.

In another aspect of the present invention, the cannabinoids include CBCA, CBDVA, CBGA, CBNA, and THCVA in carboxylated forms, and predominately decarboxylated forms of CBDA and THCA.

In another aspect of the present invention, the ratio of at least 1:20 of carboxylated to decarboxylated cannabinoids for CBDA:CBD is maintained in manufactured animal feeds and human foods made with a cold or hot-extrusion process.

Further in another aspect of the present invention is a method for manufacturing pelletized animal food having safe levels of Cannabidiol, the method includes providing a food substrate having cannabinoids, and a combined cannabidiol (CBD) and cannabidiolic acid CBDA) concentration of less than 5 mg/kg of the food substrate; pelletizing the substrate at a maximum temperature of no more than 120° C. to preserve cannabidiolic acid (CBDA) in its carboxylated form; and cooling the pelletized substrate to an ambient temperature wherein the pelletized substrate is animal food having a moisture content of less than 20%.

In some aspects of the present invention, the industrial hemp includes chlorophyll-containing portions of the hemp plant including leaves and flowers.

It will be understood that certain ingredients can be added to the compositions described herein without materially affecting the basic and novel properties of the compositions described herein. For example, the compositions can include undisclosed and/or unclaimed ingredients that do not materially affect the basic and novel properties of the compositions described herein, therapeutic or otherwise. Other variations, embodiments and features of the present disclosure will become evident from the following detailed description, abstract and claims.

### BRIEF DESCRIPTION OF THE DRAWINGS

The object of the invention may be understood in more details and more particularly description of the invention briefly summarized above by reference to certain embodiments thereof which are illustrated in the appended drawings, which drawings form a part of this specification. It is to be noted, however, that the appended drawings illustrate preferred embodiments of the invention and are therefore not to be considered limiting of its scope, for the invention may admit to other equally effective equivalent embodiments.

FIG. 1 shows a formulation process of animal feed rations including cannabinoids in an amount related to the Daily Metabolizable Energy Requirements (DMER) of the animal in accordance with the embodiments of the present invention;

FIG. 2 shows a graphical representation of the feed intake by body weight (BW) of the animal and formulation in accordance with the embodiments of the present invention;

FIG. 3 shows another graphical representation of the feed intake variance based on formulation in accordance with the embodiments of the present invention;

FIG. 4 shows extruded animal feed ration manufacturing process in accordance with the embodiments of the present invention;

FIG. 5 shows an extrusion system in accordance with the embodiments of the present invention; and

FIG. 6 shows a graphical representation of Material changes caused by pressure and temperature in accordance with the embodiments of the present invention.

### DETAILED DESCRIPTION

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

### LIST OF ABBREVIATIONS

### Δ8-THC Δ8 Tetrahydrocannabinol

### AAFCO Association of American Feed Control Officials

AOAC The Association of Official Agricultural Chemists (a.k.a. AOAC International)

### AOCS The American Oil Chemists' Society

### CBC Cannabichromene

CBCA Cannabichromenic acid

### CBD Cannabidiol

CBDA Cannabidiolic acid

### CBDV Cannabidivarin

CBDVA Cannabidivarinic acid

### CBG Cannabigerol

CBGA Cannabigerolic acid

### CBL Cannabicyclol

### CBN Cannabinol

CBNA Cannabinolic acid

### CFR Unites States Code of Federal Regulations

CI Confidence interval for an observed mean

### COA Certificate of Analysis

CPHSO Cold-Pressed Hemp Seed Oil (mechanically extracted at <50° C.)

### GC Gas Chromatography

GLP Good Laboratory Practices (as defined by 21 CFR § 58)

### LC Liquid Chromatography

LOD Level of detection specifies the minimum amount of detectable analyte
LOQ Level of quantitation specifies the minimum amount of quantifiable analyte
ME Metabolizable energy

### MS Mass Spectrometry

### NRC National Research Council of the National Academies

ppb Parts per billion
ppm Parts per million
RER Resting energy required (or requirement)

### THC Δ9-Tetrahydrocannabinol (Δ9-THC)

THCA Δ9-Tetrahydrocannabinolic acid (Δ9-THCA)

### THCV Tetrahydrocannabivarin

THCVA Tetrahydrocannabivarinic acid
UHPLC-MS/MS Ultra High-Performance LC with MS/MS Detection
w/w Weight to Weight (dry weight basis)

The present invention provides a method for safe and effective incorporation of ingredients derived from the harvested seeds of the hemp (Cannabis Sativa L) plant by limiting the cannabinoid intake to less than 1.2 mg/kg of animal body weight (<1.2 mg/kgBW) per 24-hour period through animal feed ration (dietary formulation) incorporating mathematically calculated maximum exposures based on cannabinoid concentrations in the source ingredients.

In one embodiment, the animal feed ration produced by the methods of the present invention have a relative concentration of CBDA:CBD at 1:20 to achieve bioactivity such as a reduction in inflammation, tumor growth inhibition, pain management, and other adverse biological issues, conditions or chronic disease. Including this relative amount of CBDA has yielded improved results in various contexts described herein.

### Formulation

The formulation process starts with the selection of the target species such as cat, dog, horse, etc. Each species has specific nutritional and energy requirements that must be satisfied in the dietary formulation. When incorporating cannabis, the formulation process becomes an iterative (mathematical, physical, or theoretical) exercise to limit the cannabinoid content to a maximum level, or to achieve a specific content in the animal feed ration.

Formulators, nutritionists, regulators, and veterinarians determine the recommended or required minimum and maximum animal energy and nutrient requirements, accordingly the energy requirement is adjusted in the formulation process. It should be noted that at least two different equations are used to calculate the fulfillment of basic metabolizable energy (ME) requirements. These, and other formulas, are primarily used to calculate the energy required per kilogram of body weight, which is a non-linear equation for almost every species.

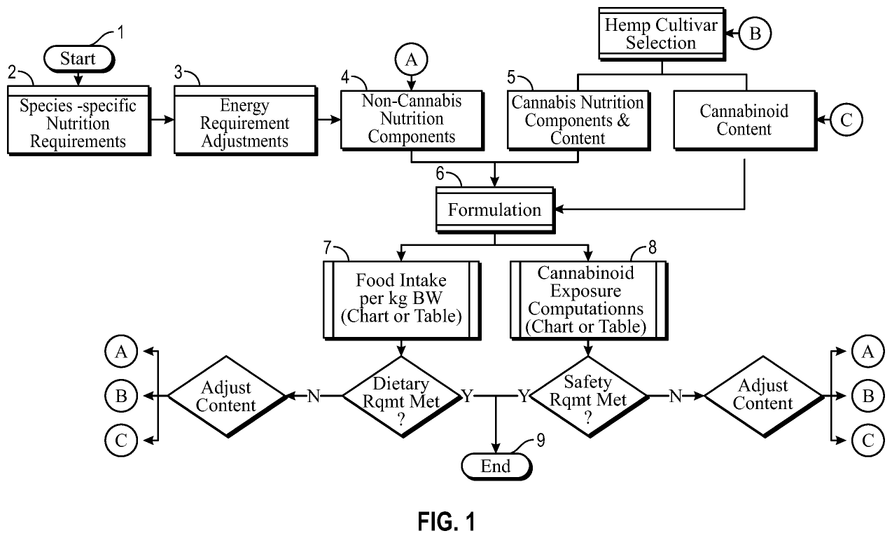

FIG. 1 demonstrates an example of a formulation process for yielding an appropriate cannabinoid for mixing or adding to a daily animal feed ration. In an exemplary embodiment, the formulation process is described herewith various steps with reference to the FIG. 1. The formulation process starts with the selection of the target species (animals), (1), then, the species, age and breed specific nutrition requirements are determined for a particular class or classes of animals, (2) and the daily metabolizable energy requirement (DMER) is determined for the particular class or classes of animals, (3). After that the nutritional components (4) particularly non-cannabis components (A) are added to a feed substrate, and also the Cannabis nutritional components/contents (5) are added to the feed substrate. Further, Cannabinoid contents (C) are added to form a formulation (6). Whereas, the selection of the Cannabis nutritional components (5) and Cannabinoid content (C) are basically influenced by the Hemp cultivar selection (B) and can vary greatly depending on the ingredient and source cultivar. Once the above-described components/contents are added to form the formulation (6), the formulation process undergoes with two other steps for ensuring dietary requirement and safety of the formulation (6). These are adjusting the formulation (6) based on the food intake per kg BW (chart or table) (7) and cannabinoid exposure computation (chart or table) (8). The food intake per kg BW (chart or table) (7) determines the dietary requirement, if the dietary requirement is met, the formulation process completes at step (9), if the dietary requirement is not met, then the components/contents of (A), (B) and (C) are adjusted. Similarly, the cannabinoid exposure computation (chart or table) (8) determines the safety requirements, if the safety requirement is met, the formulation process completes at step (9), if the safety requirement is not met, then the components/contents of (A), (B) and (C) are adjusted.

In some embodiments of the present invention, the manufacturing process extrudes or pelletizes the feed substrate and the nutritional components as well as the cannabinoids. Carboxylated cannabinoids are generally preferred by dietary experts as these are the naturally occurring predominant form found in the plant. The formulation process of the present invention is biased towards maximizing the carboxylated cannabinoids when compared to the decarboxylated forms of the particular cannabinoid molecules.

While the present invention is described herewith by way of example, the description is not intended to limit the invention. Further, as the invention is explained in terms of CBD usage primarily, other cannabinoids or combinations of cannabinoids can be substituted for CBD in accordance with the present invention. The amounts of the other cannabinoids, or combinations thereof, may collectively equal the concentrations of the CBD in one embodiment of the present invention.

In the embodiments of the present invention, commonly used formulas and methods for determination of the daily metabolizable energy requirement (DMER) include the National Research Council of the National Academies (NRC) recommendations and the Association of Feed Control Officials (AAFCO) recommendations. Both methods have formula factors that are adjusted based on the species. The examples contained in this document include dogs and cats. References for other species are readily available from AAFCO and the NRC.

These are two major ways of computing energy requirements in animals, such as dogs. One is the NRC method and the other is the AAFCO method. The NRC is the National Resource Counsel of the National Academy of Sciences. The AAFCO is the Association of Animal Feed Control Officials. These equations have been developed to express the minimum feeding requirements for animals, including dogs. The present invention uses at least one of these equations and methods to determine the energy intake requirement of an animal. Based on this energy intake requirement, a properly balanced and safe cannabinoid content delivered on a daily basis is computed and delivered to the particular animal, or class of animals.

The National Research Council of the National Academies (NRC) method recommends using Equation 1 for adult dogs. The Association of Feed Control Officials (AAFCO) recommends Equation 2. Both equations are commonly used and will produce identical results with the proper input parameters.

Equation 1: NRC energy requirement computation for adult dogs

DMER (inkcals)=RER_{MUL}*70*(kgBW)^{-0.75}
where,
RER_{MUL} is a multiplicative adjuster for body condition and other factors which ranges from 1.0 to 5.0 (or more), and
kgBW is the animal body mass expressed in kilograms.

Equation 2: AAFCO Energy Requirement Computation for Adult Dogs

DMER (in kcals)=130*(kgBW)^{-0.75}
where,
kgBW is the animal body mass expressed in kilograms

Further, for adult domesticated cats, the NRC recommends Equation 3, while AAFCO recommends Equation 4. The NRC and AAFCO provide various formulation guidance and adjustments for dogs and cats based on a 4000 kcal ME/kg target diet, but commercially manufactured animal feeds frequently contain more or less than the 4000 kcals ME/kg energy content depending on their ingredients and form (dry extruded, semi-moist, wet, canned, etc.).

Equation 3: NRC Energy Requirement Computation for Adult Cats

DMER(in kcals)=RER_{MUL}*70*(kgBW)^{-0.67}
where,
RER_{MUL} is a multiplicative adjuster for body condition and other factors which ranges from 1.0 to 5.0 (or more), and
kgBW is the animal body mass expressed in kilograms.

Equation 4: AAFCO Basic Energy Requirement Computation for Adult Cats

DMER (in kcals)=100*(kgBW)^{-0.67}
where,
kgBW is the animal body mass expressed in kilograms.

Using an RER_{MUL} of 1.85715 in Equation 1 yields virtually the exact same result (give or take a few decimal places) as using Equation 2. Similar results are achieved with either formula, and both provide a suitable outcome for formulation computations.

For a weight loss diet, NRC recommends a variety of RER_{MUL} factors, like 1.0 or 1.2 while AAFCO recommends formulating the diet with lower nutrient content (<3100 kcal ME/kg for low-moisture (dry) dog foods and <3250 kcal ME/kg for dry cat foods).

Conversely, a higher RER_{MUL} is used for weight gain, puppies (growth cycle), working dogs, or specific species that require more daily energy, while AAFCO recommends formulating a higher-energy diet.

In some embodiments the invention provides a wide variety of formulations of different nutritional content, and can be exploited into “breed-specific” formulations and brands.

In some embodiments, basic energy and nutrition requirements are further adjusted for specific breeds or use, body condition (lean, normal or overweight), life-cycle (adult, breeding, senior, intact or neutered), and dietary purpose (weight sustaining, loss/gain, high-energy, etc.).

Available sources of proteins (beef, chicken, and fish), plants (fruits and vegetables), carbohydrates, fates, vitamins, and minerals form the basic formulation. These are the primary factors in the selection of non-cannabis nutrition components that comprise a critical portion of the formulation and determine the input mixtures of ingredients containing fats, proteins, carbohydrates, free nitrogen content, etc.

### Feeding Quantity Computations

The ingredients selection and the nutritional formulation will determine the final Metabolizable energy (ME) in the animal feed. Charts and tables of the required daily food intake (amount of food the animal is to be fed every day) are constructed based on the formula used for internal computations and energy per mass unit in the proposed diet for the intended species.

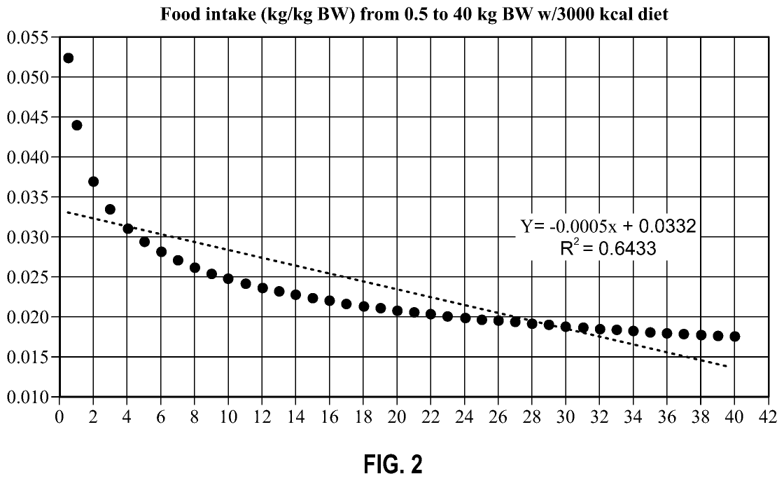

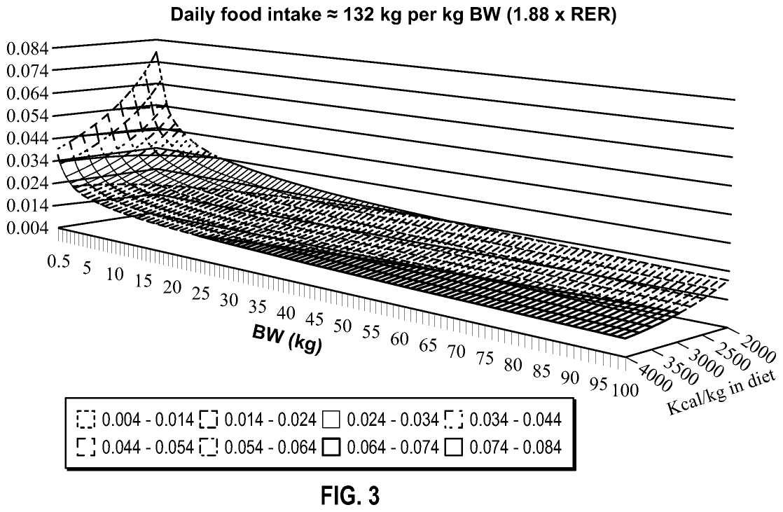

Table 1 shows a partial listing of daily feed intake chart for a hypothetical 2000-kcal (kilocalorie) dog food diet. These formulae can be used to construct models of the typical feed intake rates, or amount of food the animal needs to eat every day as shown in FIG. 2 and FIG. 3. Feed intake tables/charts can be pre-constructed for a wide range of energy content (kcal/kg) diets and used as a reference point when starting the formulation process.

TABLE 1
Partial daily food intake chart for 2000 kcal dog food diet

| Body | | | | RER_{Exp} | Hemp |
|---|---|---|---|---|---|
| Weight | Diet | | | .75 | Oil |
| BW in | (kcal/kg) | RER | | Food | (w/w) |
| kgs & lbs | 2,000 | (kcals) | RER_{Mul} | Intake | 5% |
| Body | Body | 70 | 1.885 | (kg/kg | Food |
| Weight | Weight | RER = | MER = | for 2,000 | Intake |
| (BW) | (BW) | 70 × | RER_{Mul} X | kcal/kg | (kg/day) |
| in kgs | in lbs | (kg \hat{ }.75) | RER | diet | BW/day) |
| 0.5 | 1.10 | 41.6 | 78.5 | 0.0392 | 0.0785 |
| 1 | 2.20 | 70 | 132.0 | 0.0660 | 0.0660 |
| 2 | 4.41 | 117.7 | 221.9 | 0.1110 | 0.0555 |
| 3 | 6.61 | 159.6 | 300.8 | 0.1504 | 0.0501 |
| 4 | 8.82 | 198.0 | 373.2 | 0.1866 | 0.0467 |
| 5 | 11.02 | 234.1 | 441.2 | 0.2206 | 0.0441 |
| 6 | 13.23 | 268.4 | 505.9 | 0.2529 | 0.0422 |
| 7 | 15.23 | 301.2 | 567.8 | 0.2839 | 0.0406 |
| 8 | 17.64 | 333.0 | 627.7 | 0.3128 | 0.0392 |
| 9 | 19.84 | 363.7 | 685.6 | 0.3428 | 0.0381 |
| 10 | 22.05 | 393.6 | 742.0 | 0.3710 | 0.0371 |
| 11 | 24.25 | 422.8 | 797.0 | 0.3985 | 0.0362 |
| 12 | 26.46 | 451.3 | 850.7 | 0.4254 | 0.0354 |
| 13 | 28.66 | 479.2 | 903.4 | 0.4517 | 0.0347 |
| 14 | 30.86 | 506.6 | 955.0 | 0.4775 | 0.0341 |
| 15 | 33.07 | 533.5 | 1000.7 | 0.5029 | 0.0335 |
| 16 | 35.27 | 560.0 | 1055.6 | 0.5278 | 0.0330 |
| 17 | 37.48 | 586.1 | 1104.7 | 0.5524 | 0.0325 |
| 18 | 39.68 | 611.7 | 1153.1 | 0.5765 | 0.0320 |
| 19 | 41.89 | 637.0 | 1200.8 | 0.6004 | 0.0316 |
| 20 | 44.09 | 662.0 | 1247.9 | 0.6240 | 0.0312 |
| 21 | 46.30 | 686.7 | 1294.4 | 0.6472 | 0.0308 |
| 22 | 48.50 | 711.1 | 1340.4 | 0.6702 | 0.0305 |
| 23 | 50.71 | 735.2 | 1385.8 | 0.6929 | 0.0301 |
| 24 | 52.91 | 759.0 | 1430.8 | 0.7154 | 0.0298 |
| 25 | 55.12 | 782.6 | 1475.2 | 0.7376 | 0.0295 |
| 26 | 57.32 | 806.0 | 1519.3 | 0.7596 | 0.0292 |
| 27 | 59.52 | 829.1 | 1562.9 | 0.7815 | 0.0289 |
| 28 | 61.73 | 852.1 | 1606.1 | 0.8031 | 0.0287 |
| 29 | 63.93 | 874.8 | 1649.0 | 0.8245 | 0.0284 |
| 30 | 66.14 | 897.3 | 1691.4 | 0.8457 | 0.0282 |

### Cannabinoid Content Computations

It is known that there are more than 100 cannabinoids found in cannabis, however, only a handful (about sixteen cannabinoids) commonly quantified where commercial laboratory reference standards are readily available. Cold extrusion, below 80° C., will not impact the product's initial (“as manufactured”) major cannabinoid content because none of the commonly measurable cannabinoids readily decarboxylate below 80° C.

The selection of the cannabis cultivar (B) strongly influences the cannabinoid content in the cannabis ingredient (seed oil, seedcake, meal, etc.). Cannabinoid content can vary greatly depending on the ingredient and source cultivar. Not every cultivar is suitable for every application or formulation due to variance in cannabinoid content, which is primarily influenced by cultivar. Adjusting the amount of cannabis-derived ingredients included in the diet will also influence overall cannabinoid content in the feed or food product. This interaction of components creates additional complexity and iterations to the formulation process, particularly when considering the effects of decarboxylation.

The final cannabinoid content can be composed of both Carboxylated (acidic form) and decarboxylated cannabinoids. For example, both CBCA and CBC, CBDA and CBD, CBGA and CBG, and THCA and THC, among other acidic/non-acidic cannabinoid pairs, may be present. The formulator may have to account for final cannabinoid limits with or without decarboxylation, where Carboxylated acid forms shed a CO2 atom and convert to decarboxylated forms (CBDA transform to CBD, THCA transforms to THC, etc.). A computation when 100% of the Carboxylated cannabinoid is estimated to decarboxylated is shown in Equation 5. The formulating scientist can estimate the percentage of decarboxylation, or this can be tested in small batch production once the candidate formulation is determined.

Equation 5: Total Potential (TP) Cannabinoids with Individual Analyte Tests LC)

$$TP cannabinoid = decarboxylatedmass + carboxylatedmass \times \frac{carboxylated g / mol}{decarboxylated g / mol}$$

For Example: Computing TP CBD with inputs of 100 mg/kg CBDA and 50 mg/kg CBD

50 mg/kg of CBD carboxylated cannabinoid with mass=314.469 g/mol
100 mg/kg of CBDA decarboxylated cannabinoid with mass=358.478 g/mol

$$TPCBD = 50 + 100 \times \frac{314.469}{358.478} \approx 137.7 \frac{mg}{kg}$$

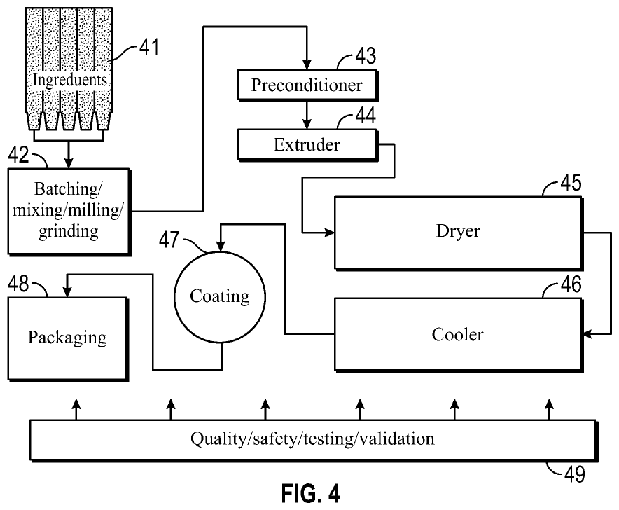

Decarboxylation can also occur under high-temperature drying conditions or some higher-temperature pelletizing operations, but hot extrusion is a primary concern. Enzymatic decarboxylation occurs naturally over time but is greatly accelerated when the carboxylated atoms are exposed to varying higher temperatures (depending on the cannabinoid), which occurs in the hot extrusion process used to make animal feed as shown in FIG. 4. The decarboxylation effects on carboxylated, decarboxylated, and total potential cannabinoids may be estimated based on extrusion time and temperature, and measured in test runs for compliance.

Feeding tables/charts can also be used to contemporaneously construct cross-referenced cannabis content tables based on the type and amount of cannabis-derived ingredients incorporated in the final formulation and the expected cannabinoid concentration in the cannabis ingredient. If the desired amounts of cannabinoid is not achieved, or a required maximum is exceeded, then adjustments in cannabinoid-containing ingredient content, or the ingredient source cannabis cultivar, and reformulation is required as described in FIG. 1. The same logic applies to any computed total potential cannabinoids, so if the desired total potential is not achieved, or any maximum total potential is exceeded, then reformulation is required.

For example, the formulator may incorporate hemp seed oil for the nutritional value, which is mostly Essential Fatty Acids (EFAs) with some minor vitamin and mineral content. Alternatively, hemp seedcake may be used for its protein content. In addition, the hemp ingredient may contain various amounts (or target values) of cannabinoids, so a relationship exists between the non-linear feeding charts (as shown in FIG. 2 and FIG. 3) and feeding quantities (as shown in table 1) and the cannabinoid content. Such cannabinoid content tables, based on the quantity of daily feed and amount of cannabinoid-containing ingredient used in the animal feed.

### Hot Extrusion Manufacturing Considerations

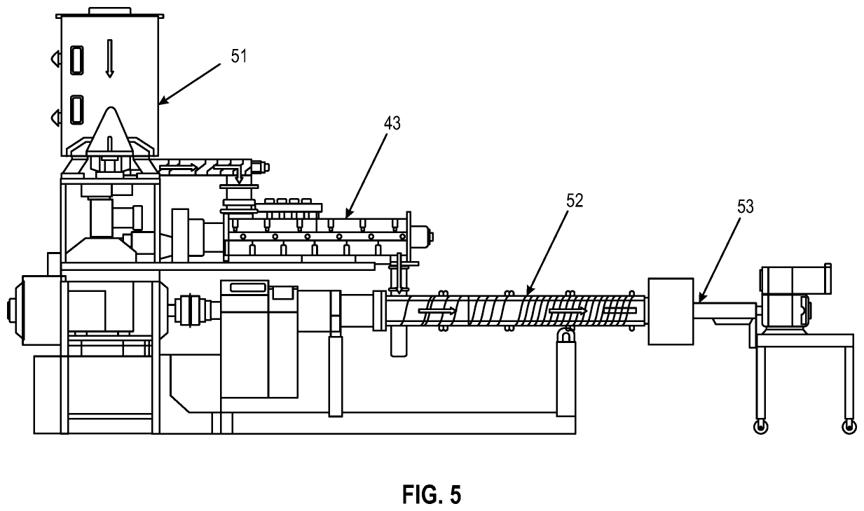

FIG. 4 and FIG. 5 shows the hot extrusion manufacturing process using the extrusion system. The hot extrusion manufacturing process starts with selecting Raw materials of ingredients (41) that continuously flow through the extrusion system from the input sources, where the “Raw materials” does not imply uncooked ingredients. The ingredients (41) at inputs can be rendered meats, dry goods, grains, or any other combination of food ingredients. These ingredients (41) can be wet and dry materials that are often mixed separately which is refereed as batching/mixing/milling/grinding (42) and then combined just before passing through the preconditioner (43) into the extruder system (44). However, in some embodiments, at smaller scales of production, wet and dry materials can be mixed simultaneously then fed into the extruder system (44) directly. After preconditioning the ingredients, it passed through the dryer (45) and then through the cooler (46). Once the ingredients are cooled, in some embodiments, coating (47) can be performed and then it is packaged (48) in a container/bag. Whereas in each step quality control (quality/safety/testing/validation) (49) is performed for ensuring the safe levels of cannabinoids in the animal feed ration.

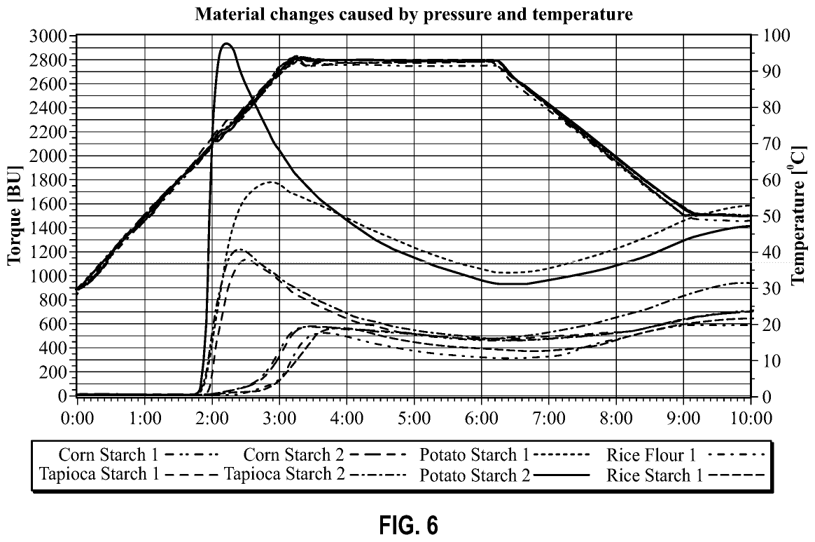

The temperature at each stage of the process and the time spent in each processing step will impact the final decarboxylated cannabinoid content. Typical time and temperature ranges for hot extruded animal feeds are shown below in Table 2, these numbers may vary depending on the quantity and types of ingredients and size of the hot extrusion manufacturing process as shown in the FIG. 6.

TABLE 2

| | | Extruder | | Cooling & |
|---|---|---|---|---|
| Preconditioner | | Barrel | Dryer | Coating |
| Low | 0.5 mins @ | 3 mins @ | 10 mins @ | 20 mins @ < |
| | 80° C. | 140° C. | 80° C. | 80° C. |
| High | 3.0 mins @ | 7 mins @ | 20 mins @ | 20 minutes @ < |
| | 80° C. | 140° C. | 80° C. | 80° C. |

In the embodiments of invention, the preconditioner helps starches to gelatinize first by doing mechanical damaged to break up the materials and adding water and steam moisture. The temperatures in the preconditioner phase are generally around 80° C. (≈176° F.). Time in the preconditioner depends on the type of ingredients and the chemical and physical properties of the input starches.

In the embodiments of the invention, the Dry ingredients in the formula are “preconditioned” before transfer into the extruder system. The step is where the initial heat and moisture are added and subsequently mixed with the ration to begin the cooking process of the starches (gelatinization). This is important for the conversion of starch from a tightly packed granule to a less ordered structure. Time in the extruder is influenced by the starches in the ingredients.

Additionally, some operations add meat slurries and fats to the preconditioner. Mixing large proportions of meat slurry, which is high in moisture and fat, requires metering of the slurry at the perfect rate relative to the dry portion and a large amount of energy and shear to maintain a consistent blend. Most materials pass through the preconditioning step relatively quickly, but the time can range from about 30 to 240 seconds (3 minutes) depending on the formulation. The preconditioning time is also influenced by the desired characteristics of the final output product (size, density, post-extrusion expansion, etc.).

As shown in the FIG. 5, the ingredients are mixed into the bin/container (51) and passed through the preconditioner (43). The preconditioner (43) output feeds directly into the extrusion barrel (52) input where pressure and temperature are quickly increased to simultaneously free polymers and cook the ingredients. Additional moisture (steam) is added to assist in the cooking process in the extruder barrel (52). The ingredient temperature can reach 110-140° C. (≈230-284° F.), depending on the application.

Pressure increases in the extruder barrel (52) and the output pressure can range greatly depending on the application. For dry pet food kibbles, pressures can reach forty (40) atmospheres, but a useful range is 150-600 kPa. The high temperatures and high pressures in the extruder barrel align the cooked polymers at the output of the extrusion barrel, but can also accelerate decarboxylation of some cannabinoids.

Cooked ingredient materials exiting the extruder barrel (53) are compressed to create a semi-solid output stream that is fed into a shaping die and cut into strips, chunks, kibbles, or other forms by the Die and Knife set-up (54) attached to the opening of extruder barrel (53). The extruder die-cutter (Die and Knife set-up 54) output product is high in moisture so it is fed into the dryer (45) to reduce the moisture. Dryers (45) typically operate in the range of 60-80° C. (160-176° F.), but this is product-dependent. Product time in the dryer 45 is frequently less than ten to fifteen (10-15) minutes.

Normally, extruded products expand naturally as they exit the extruder and cool down. For some applications, the extruded output product is further exposed to very hot oils (fried) to expand the product further in a post-processing step. For dry (low-moisture) pet food kibbles, and similar animal feeds, additional drying is required to reduce the moisture content to 8-10% of dry weight to limit microbial growth.

In alternate embodiments, safe amounts of the various other cannabinoids and combinations thereof can be determined and includes in the feed rations of the present invention based on an estimated daily metabolizable energy requirement (DMER) of a particular animal or class of animals.

Preferably the cannabinoids are included and mixed with the food substrate in the form of seed, seed cake, hemp oil, or other food grade aspect of the Cannabis sativa l. plant. It is envisioned that hemp oil can be sprayed on the food substrate post extrusion, but this may introduce flavor defects to the food product. It is preferred to include the cannabinoids in a mixture with the extruded or pelletized food substrate so that the flavor will be uniform, and not significantly affected by addition of hemp or cannabinoids to the animal feed.

In a preferred embodiment of the invention, no isolated or distilled cannabinoids are sprayed on, or mixed with, the feed product of the present invention. It is most desirable to instead, include food grade ingredients that contain trace cannabinoids in amounts that are safe to the food product of the present invention. More preferably, this amount of trace cannabinoids are included in amounts determined in large part by the daily metabolizable energy requirement (DMER) for a class of animals, or particular animals for which the feed is engineered.

The present invention includes a method for using an animal feed ration to safely stimulate the endocannabinoid system of an animal. The cannabinoid amounts expressed herein are intended not to cure any chronic disease or ailment but to build the health of the animal subjects through the regular stimulation and benefits balancing of the animal subjects' endocannabinoid system. The micro-dosing schedule i.e. daily consumption and volumes are designed using the daily metabolizable energy requirement (DMER) for a class of animals, or particular animals for which the feed is engineered in order to achieve prophylactic effects to inhibit the onset of inflammation, arthritis, pain, anxiety and other conditions that cannabinoids may be understood to influence by modulating the animal endocannabinoid system.

Various examples of implementation of the present invention and methods thereof.

Example 1 for Computing CBD for an Average One-Kilogram Canine:
RME=3000 kcals/kg
DMER=130 kcals
PCI=3%
CC=50 mg/kg of CBD

$$MDCI = \frac{130 kcals}{3000 kcals} \times 3 \% \times 50 \frac{mg}{kg} = 0.195 mg CBD / day = 0.1 \; 95 \frac{mg}{kgBW} / day$$

Example 2 for an Average Five-Kilogram Household Canine:
DMER=434.7 kcals
RME=3000 kcals/kg
PCI=3%
CC=50 mg/kg of CBD

$$MDCI = \frac{434.7 kcals / kg}{3000 kcals} \times 3 \% \times 50 \frac{mg}{kg} = 0.54335 mg of CBD / day = 0.1 \; 0867 \frac{mg}{kgBW} / day$$

[Example for Computing CBD for an Average One-Kilogram Canine:
RME=3000 kcals/kg
DMER=130 kcals
PCI=3%
CC=50 mg/kg of CBD

$$MDCI = \frac{3000 kcals / kg}{130 kcals} \times 3 \% \times 50 \frac{mg}{kg} = 0.195 mg CBD / day = 0.195 \frac{mg}{kgBW} / day$$

Example for an Average Five-Kilogram Household Canine:
RME=3000 kcals/kg
DMER=434.7 kcals
PCI=3%
CC=50 mg/kg of CBD

$$MDCI = \frac{3000 kcals / kg}{434.7 kcals} \times 3 \% \times 50 \frac{mg}{kg} = 0.54335 mg of CBD / day = 0.10867 \frac{mg}{kgBW} / day ]$$

Any alterations and further modifications of the compositions and/or formulations described herein, which would normally occur to one skilled in the relevant art and having possession of this disclosure, are to be considered within the scope of the instant claims.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

I claim:

1. A method for using an animal feed ration to safely stimulate endocannabinoid system of an animal, comprising:
determining a daily metabolizable energy requirement (DMER) for a class of animals;
providing an animal feed ration meeting the daily metabolizable energy requirement (DMER) to at least one animal in said class of animals, with at least one of the animal having a body weight (BW) measurable in kilograms (kg);
the animal feed ration having cannabidiol (CBD) and cannabidiolic acid (CBDA) in a combined concentration determined in part by the daily metabolizable energy requirement (DMER), and said combined concentration being less than 1.0 mg/kg of the body weight (BW) of a mammalian subject, and said ratio of CBDA:CBD being at least 1:20; and
delivering, on a daily basis, the feed ration to the mammalian subject in an amount of 2000 to 20000 kcal/kg of the animal subject BW,
wherein, the DMER is determined by a formula:
DMER=RER_{MUL}*β(kgBW)^{-α}
where,
DMER is the Daily Metabolizable Energy Required (in calories);
RER_{MUL} is a Resting Energy Required (RER);
kgBW is the animal body mass expressed in kilograms;
α is an adjustable factor having a value between 0.25 and 1.75;
β is a value between 65-6500; and
wherein, the maximum amount of combined cannabidiol (CBD) and cannabidiolic acid (CBDA) provided is determined by a formula:
$$Maximum daily cannabinoid intake ( MDCI ) = \frac{D M E R}{R M E} \times PCI \times CC$$
where,
DMER is the daily metabolizable energy requirement for the animal;
RME is the ration metabolizable energy per unit of mass (e.g., 3000 kcal s/kg);
PCI is the percentage of cannabis ingredient in the ration on a dry weight (w/w) basis; and
CC is the cannabinoid concentration in the cannabis ingredient on a dry weight basis.

2. The method of claim 1, wherein the combined CBD and CBDA concentration is greater than 0.1 mg/kg.

3. The method of claim 2, wherein the animal feed ration includes hemp oil that causes the CBD and CBDA concentration to be greater than 0.1 mg/kg.

4. The method of claim 3, wherein the animal feed ration includes hemp oil in a concentration of no more than 6% on a w/w basis in the animal feed ration.

5. The method of claim 1, wherein the ratio of CBDA:CBD in at least 1:20 and the animal feed ration is produced by extrusion.

6. The method of claim 1, wherein the ratio of CBDA:CBD in at least 1:20 and the animal feed ration is produced by pelletization.

7. The method of claim 1, wherein the animal feed ration is a canine feed ration having an absorbable energy of 2000-5000 kcal/kg

8. The method of claim 1, wherein the animal feed ration is equine feed ration having an absorbable energy of 10,000-15000 kcal/kg.

9. The method of claim 1, wherein the animal feed ration is feline feed ration having an absorbable energy of 2000-5000 kcal/kg.

10. The method of claim 1, wherein the animal fee ration has a ratio of CBDA:CBD of at least 1:20 to inhibit hepatoxicity in the at least one animal.

11. The method of claim 1, wherein the class of animals is chosen from the group consisting of canines, equines, felines, or other mammalians.

12. The method of claim 11, wherein the class of animals is subdivided by age.

13. The method of claim 1, wherein the class of animals is further defined by breed, class, age, body condition or other factors.

14. A method for manufacturing extruded animal food having safe levels of cannabinoids, comprising:
providing a food substrate having a cannabinoid content of a combined cannabidiol (CBD) and cannabidiolic acid (CBDA) concentration of less than 5 mg/kg of the food substrate;
extruding the substrate at a maximum temperature of no more than 145° C. and a pressure of no more than 50 kg/cm²; and
cooling the extruded substrate to ambient temperature, wherein the extruded substrate is animal food having a moisture content of less than 35%, a bulk density of between 150-600 g/l, and at least 5% of the total Cannabinoid content is the cannabidiolic acid (CBDA).

15. The method as set forth in claim 14, wherein no isolated cannabidiol or cannabidiolic acid is added to the food substrate before or after the step of cooling.

16. The method as set forth in claim 14, wherein no distilled cannabidiol or cannabidiolic acid is added to the food substrate before or after the step of cooling.

17. The method as set forth in claim 14, wherein no concentrated cannabidiol or cannabidiolic acid is added to the food substrate before or after the step of cooling.

18. The method as set forth in claim 14, wherein the step of providing a food includes providing cannabidiol (CBD) and cannabidiolic acid (CBDA) from Cannabis sativa L.

19. The method as set forth in claim 18, wherein the Cannabis sativa L. is industrial hemp, having less than 0.3% tetrahydrocannabinol content on a dry weight (w/w) basis.

20. The method as set forth in claim 18, wherein the industrial hemp is made from the hemp components selected from the group consisting of crushed hemp seeds, de-hulled hemp seed, hemp seed meal, hemp seed cake, hemp seed oil, or combinations thereof.

21. The method as set forth in claim 18, wherein the industrial hemp is only seed meal.

22. The method as set forth in claim 18, wherein the industrial hemp is only hemp seed oil.

23. The method as set forth in claim 18, wherein the industrial hemp includes chlorophyll-containing portions of the hemp plant, including leaves and flowers.

24. The method as set forth in claim 14, wherein the animal food is achieved through formulation computations to limit the daily intake and exposure to any individual cannabinoid analyte to a maximum of 1.5 milligrams per kilogram of body weight per day (mg/kg BW/day) in animal food.

25. The method as set forth in claim 14, wherein the formulation limits the daily intake and exposure to any total potential decarboxylated cannabinoid analyte group (e.g., CBC, CBD, CBDV, CBG, CBL, CBN, THC, THCV) to a maximum of 1.5 milligrams per kilogram of body weight per day (mg/kg BW/day) in animal feeds.

26. The method as set forth in claim 14, wherein the cannabinoid includes two or more of CBCA, CBC, CBDA, CBD, CBDVA, CBDV, CBGA, CBG, CBL, CBNA, CBN, THCA, THC, THCVA, and THCV, and at least 3% of the total cannabinoid mass (in mg/kg or μg/g) of the sum of CBCA, CBC, CBDA, CBD, CBDVA, CBDV, CBGA, CBG, CBL, CBNA, CBN, THCA, THC, THCVA, and THCV consists of the cannabinoid CBDA in the manufactured animal feed or human food product.

27. The method as set forth in claim 14, wherein cannabinoids include CBCA, CBDVA, CBGA, CBNA, and THCVA in carboxylated forms, and predominately decarboxylated forms of CBDA and THCA.

28. The method as set forth in claim 14, wherein a ratio of at least 1:20 of carboxylated to decarboxylated cannabinoids for CBDA:CBD is maintained in manufactured animal feeds and human foods made with a cold or hot-extrusion process.

29. A method for manufacturing pelletized animal food having safe levels of Cannabidiol, comprising:
providing a food substrate having cannabinoids, and a combined cannabidiol (CBD) and cannabidiolic acid CBDA) concentration of less than 5 mg/kg of the food substrate;
pelletizing the substrate at a maximum temperature of no more than 120° C. to preserve cannabidiolic acid (CBDA) in its carboxylated form; and
cooling the pelletized substrate to an ambient temperature wherein the pelletized substrate is animal food having a moisture content of less than 20%.

30. The method as set forth in claim 29, wherein no isolated cannabidiol or cannabidiolic acid is added to the food substrate before or after the step of cooling.

31. The method as set forth in claim 29, wherein no distilled cannabidiol or cannabidiolic acid is added to the food substrate before or after the step of cooling.

32. The method as set forth in claim 29, wherein no concentrated cannabidiol or cannabidiolic acid is added to the food substrate before or after the step of cooling.

33. The method as set forth in claim 29, wherein the method of providing a food includes providing Cannabidiol and cannabidiolic acid from Cannabis sativa L.

34. The method as set forth in claim 29, wherein the Cannabis sativa L. is industrial hemp, having less than 0.3% tetrahydrocannabinol content on a dry weight w/w basis.

35. The method as set forth in claim 34, wherein the industrial hemp is made from the hemp components selected from the group consisting of crushed hemp seeds, de-hulled hemp seed, hemp seed meal, hemp seed cake, hemp seed oil, or combinations thereof.

36. The method as set forth in claim 34, wherein the industrial hemp is only seed meal.

37. The method as set forth in claim 34, wherein the industrial hemp is only hemp seed oil.

38. The method as set forth in claim 34, wherein the industrial hemp includes chlorophyll-containing portions of the hemp plant including leaves and flowers.

39. The method as set forth in claim 34, wherein the industrial hemp includes chlorophyll-containing portions of the hemp plant including leaves and flowers.
